(19) United States
(12) Patent Application Publication
(10) Pub. No.: US 2010/0286379 A1
(43) Pub. Date: Nov. 11, 2010

(54) BISULPHITE TREATMENT OF RNA

(75) Inventors: Douglas Spencer Millar, New South Wales (AU); John R. Melki, New South Wales (AU)

Correspondence Address: KNOBBE MARTENS OLSON & BEAR LLP, 2040 MAIN STREET, FOURTEENTH FLOOR, IRVINE, CA, 92614, US

(73) Assignee: HUMAN GENETIC SIGNATURES PTY LTD., North Ryde, NSW (AU)

(21) Appl. No.: 12/744,310

(22) PCT Filed: Dec. 4, 2008
(86) PCT No.: PCT/AU08/01796

§ 371 Date: Jul. 27, 2010

(30) Foreign Application Priority Data
Dec. 5, 2007 (AU) 2007906651

(51) Int. Cl. C07H 21/02

(52) U.S. Cl. 536/23.1

(57) ABSTRACT

The invention relates to a method for bisulphite treating RNA comprising reacting RNA with a bisulphite reagent at 50-90° C. for 5-180 minutes so as to form treated RNA and recovering the treated RNA.

### TECHNICAL FIELD

The present invention relates to methods for treating RNA using bisulphite.

### BACKGROUND ART

It has been demonstrated that, in single stranded DNA, sodium bisulphite preferentially deaminates cytosine to uracil, compared to a very slow rate of deamination of 5-methylcytosine to thymine (Shapiro, R., DiFate, V., and Welcher, M, (1974) J. Am. Chem. Soc. 96: 906-912). This observation served as the basis for the development of the bisulphite genomic sequencing protocol of Frommer et al 1992 [Frommer M, McDonald L E, Millar D S, Collis C M, Watt F, Grigg G W, Molloy P L and Paul C L. PNAS 89: 1827-1831 (1992), which is incorporated herein by reference]. In summary, the Frommer method as presently practiced involves the following general steps: alkaline denaturation of DNA; deamination using sodium bisulphite; desuiphonation by desalting followed by alkali treatment and neutralization. Although such conditions are suitable for the bisulphite treatment of DNA, RNA would be totally destroyed by the harsh conditions used. Thus, any assay that was to utilise sodium bisulphite treatment of RNA under these conditions would be useless in a clinical environment due to degradation.

One of the major disadvantages of the bisulphite modification procedure, even using DNA as a starting material, and the established variation thereof is that it has been shown that the procedure results in the degradation of between 84-96% of the original input DNA (Grunau et al. Nucleic Acids Research 29 (13) e65 (2001)). The high loss associated with the procedure means that practically it is very difficult to successfully analyse small numbers of cells for their methylation status, or successfully analyse ancient archival specimens in which the DNA is already in a partially degraded state. In addition, due to inherent degradation associated with the current methods, it is not possible to sequence and assemble the complete genome of an organism to determine its genome-wide methylation profile in the same manner as has been successfully applied by the public Human Genome Project (International Human Genome Sequencing Consortium, 2001, Nature, 409, 860-921) or the private CELERA sequencing project (J Craig Venter et al., 2001, Science, 291, 1304-1351) as the DNA would be so fragmented that it would not be able to be cloned, sequenced, and assembled in any meaningful way owing to the huge number of “gaps” in the sequence. As can be appreciated from the above observations such conditions would produce complete degradation of RNA, thus preventing any downstream procedure for detecting.

A further disadvantage with the bisulphite method as presently practiced is that, in general, only small fragments of DNA can be amplified. Experience shows that generally less than about 500 base pairs (bp) can be successfully treated and amplified. The present technique is not applicable to new molecular biological methods such as Long Distance polymerase chain reaction (PCR) which has made it possible to amplify large regions of untreated genomic DNA, generally up to about 50 kb. At present, it is not even possible to analyse the methylation status of intact genes, as a large number of genes in mammalian genomes exceed 50 kb in length. Again the amplification of bisulphite treated RNA would be further compounded by the above facts.

To look at the methylation status of even relatively small genes (<4 kb), PCR reactions have had to be staggered across the gene region of interest (D. S Millar, K. K Ow, C. L. Paul, P. J. Russell, P. L. Molloy, S. J. Clarke, 1999, Oncogene, 18(6):1313-24; Millar D S, Paul C L, Molloy P L, Clarke S J. (2000). J Biol Chem; 275(32):24893-9). The methods presently used for bisulphite DNA treatment have also been laborious and time consuming. Standard methods typically require multiple tube changes, column purifications, dialysis, embedding the DNA in agarose beads or the addition of additives to the reaction in an attempt to reduce problems such as non-conversion of certain regions of genomic DNA.

Due to the difficulties described above, there has been no standard method for bisulphite treatment of RNA developed to date. Shapiro et al. (Shapiro R, Cohen B I and Servis R E, 1970, Nature, 227: 1047-8) first described the use of bisulphite on RNA as a tool to study the structure and function of RNA. However, very large quantities of RNA were used (5 mg) and the procedure was only 92% efficient even after incubation with the bisulphite reagent for 7 days. Others have tried to utilise bisulphite treatment of RNA to determine tertiary structure of RNA (Lowden et al., 1976, Nucl. Acids. Res. 76:3383-96; Goddard et al., 1978, 89:531-47; Digweed et al., 1982, 127:531-37) but these methods result in very little conversion of the cytosines to uracils within the nucleic acid. Subsequently, Mellor et al., (Mellor E J C, Brown F and Harris T J R, 1985, J Gen Virol, 66:1919-29) has described a method for bisulphite conversion of RNA, but only 60-80% of the cytosines were modified in a complicated process that took at least 120 hours to complete and required 2.5 μg of starting RNA.

The present inventors have previously developed methods for bisulphite treating nucleic acids. Such methods are disclosed in U.S. Pat. No. 7,288,373, which describes bisulphite treatment of DNA, and WO 2004/096825, and are commercially available in kit form under the trademark Methyleasy™.

Other commercially available kits for sulphite treating DNA are sold under the trade names methyl SEQr bisulphite conversion kit (Applied Biosystems, cat #4374960), the Methylamp-96 DNA modification kit (Epigentek, cat #P-1008), the EpiTect bisulphite kit (Qiagen, cat #59104), and the EZ DNA methylation direct kit (Zymo Research, cat #D5020).

Thus, for the bisulphite treatment of RNA, and particularly for interrogation of low amounts of starting material, a more reliable method that does not lead to substantial RNA degradation and which overcomes or at least reduces one or more of the problems associated with known RNA treatment, is required.

Through extensive investigation and research, the present inventors have now developed a robust assay for the bisulphite treatment of RNA, which results in minimal or no degradation of the RNA and which enables bisulphite treatment, analysis and recovery of extremely small RNA samples.

### DISCLOSURE OF INVENTION

The present invention relates to an improved method for bisulphite treatment of RNA which is efficient, adaptable for use with many different molecular biological techniques, and can achieve significant recovery of RNA without significant degradation.

In a first aspect the present invention provides a method for bisulphite treating RNA comprising:

reacting RNA with a bisulphite reagent at about 50-90° C. for about 5-180 minutes so as to form treated RNA; and

recovering the treated RNA.

In an embodiment of the invention the method further comprises carrying out partial or total desulphonation of the recovered RNA.

In another embodiment of the invention the method further comprises a denaturing step prior to the reacting step.

In other embodiments the method may comprise a capturing step, whereby RNA may be bound to a solid phase, such as magnetic beads. When RNA is bound to a solid phase for one or more steps, the method may also include an elution step to remove the RNA from the solid phase.

The bisulfate reagent may be sodium bisulphite or sodium metabisulphite. Preferably, the bisulphite reagent is sodium metabisulphite. Preferably, the reacting step is carried out using a bisulphite reagent at a concentration of about 1 M to about 6 M. More preferably, the concentration is about 2 M to about 4 M. In a particularly preferred embodiment the concentration of bisulphite reagent is about 3 M.

Preferably, the bisulphite reacting step is carried out for about 5-180 minutes. In a preferred embodiment, the reacting step is carried out for about 5-150 minutes. In another embodiment the reacting step is carried out for about 5-120 minutes. In a further embodiment the reacting step is carried out for about 5-90 minutes. In another embodiment the reacting step is carried out for about 5-60 minutes. In another preferred embodiment the reacting step is carried out for about 10-30 minutes. In a particularly preferred embodiment the reacting step is carried out for about 20 minutes. In another preferred embodiment the reacting step is carried out for about 30 minutes. In a further preferred embodiment the reacting step is carried out for about 45 minutes. In another preferred embodiment the reacting step is carried out for about 60 minutes. In a further preferred embodiment the reacting step is carried out for about 90 minutes. In another preferred embodiment the reacting step is carried out for about 120 minutes. In a further preferred embodiment the reacting step is carried out for about 150 minutes.

Preferably, the reacting step is carried out at a temperature of about 50° C. to about 90° C. In another embodiment the reacting step is carried out at a temperature of about 65° C. to about 85° C. In a further embodiment the reacting step is carried out at a temperature of about 60° C. to about 80° C. In various preferred embodiments the reacting step is carried out at a temperature of about 60° C., 70° C., 75° C., 80° C. or 85° C. In a particularly preferred embodiment the reacting step is carried out at a temperature of about 70° C.

In a preferred embodiment the reacting step is carried out using sodium bisulphite or sodium metabisulfite at a concentration of about 3M for about 10-30 minutes at a temperature of about 60-80° C. In an especially preferred embodiment the reacting step is carried out using sodium bisulphite or sodium metabisulfite at a concentration of about 3M for about 20 minutes at a temperature of about 70° C.

The reacting step may be carried out in the presence of an additive capable of enhancing the bisulphite reaction. The additive may be dithiothreitol (DTT), quinol, urea, methoxyamine, or mixtures thereof.

The method may further include a dilution step after the reacting step to reduce salt concentration to a level which will not substantially interfere with a nucleic acid precipitating step or binding of the treated RNA to a solid phase. The dilution step may be carried out using water to reduce salt concentration to below about 1 M, preferably, below about 0.5 M.

The recovering step may comprise precipitating the diluted treated RNA, or washing the solid support to substantially remove material, such as salts, including bisulphite salts, from the bound treated RNA. RNA precipitation may be carried out using an alcohol precipitating agent. The alcohol precipitating agent may be isopropanol, ethanol, butanol, methanol, or mixtures thereof. Preferably, the alcohol is isopropanol. The solid support may comprise magnetic beads.

Preferably, the optional denaturing step, if included in the method, is carried out using heat to denature the RNA, typically at temperatures from about 50° C. to about 90° C. More preferably, the denaturing step is carried out by heating the RNA sample to about 80° C.

Desulphonation of the recovered treated RNA may comprise removing sulphonate groups present on the treated RNA so as to obtain a treated RNA substantially free of sulphonate groups or having a reduced number of sulphonate groups, without inducing significant amounts of RNA strand breakage. The optional desulphonation step, if included in the method, may be carried out by adjusting the pH of the recovered RNA with a buffer or alkali reagent to remove some or all sulphonate groups present on the treated RNA and thereby obtain a RNA sample substantially free of sulphonate groups. Preferably, the desulphonation step is carried out at an alkaline pH of from about 7.5 to about 11.5. More preferably, the pH is from about 8.5 to about 11.5. In a preferred embodiment, the pH is about 8.7. In another preferred embodiment the pH is about 10.5. In a further preferred embodiment the pH is about 11.5.

Preferably, desulphonation is carried out at a temperature of from about 0° C. to about 90° C. In various preferred embodiments, the temperature may be in a range selected from about 5° C. to about 85° C., about 10° C. to about 70° C., about 20° C. to about 60° C., or about 30 to about 50° C. In preferred embodiments, desulphonation is carried out at a temperature of about 5° C., about 10° C., about 20° C., about 30° C., about 40° C., about 50° C., about 60° C., about 70° C., about 75° C., about 80° C. or about 85° C.

Preferably, desulphonation is carried out for about 1-30 minutes. In various preferred embodiments desulphonation is carried out for about 5-25 minutes, or about 10-20 minutes. In particularly preferred embodiments desulphonation is carried out for about 1-10 minutes, or about 5-10 minutes.

In a preferred embodiment desulphonation is carried out at a pH from about 8.5 to about 11.5 for about 1-20 minutes at about 10° C.-85° C. In another preferred embodiment desulphonation is carried out at a pH from about 8.5 to about 11.5 for about 5-15 minutes at about 20-75° C. In a further preferred embodiment desulphonation is carried out at a pH from about 8.5 to about 11.5 for about 5-15 minutes at about 20-60° C. In another preferred embodiment desulphonation is carried out at a pH from about 8.5 to 11.5 for about 5-10 minutes at about 30-50° C. In a particularly preferred embodiment desulphonation is carried out at a pH from 8.5 to 11.5 for about 5 minutes at about 40° C. In an especially preferred embodiment desulphonation is carried out at a pH of about 8.7 for 5 minutes at 40° C., or a pH of about 11.5 for about 5 minutes at 40° C.

Preferably, more than about 70% of the bisulphite treated RNA is recovered, preferably more than about 75% of the bisulphite treated RNA is recovered, preferably, more than about 80% of the bisulphite treated RNA is recovered, preferably, more than about 90% of the bisulphite treated RNA is recovered, preferably, more than about 95% of the bisulphite treated RNA is recovered.

Preferably, more than about 70% of the desulphonated RNA is recovered, preferably, more than about 75% of the desulphonated RNA is recovered, preferably, more than about 80% of the desulphonated RNA is recovered, preferably, more than about 90% of the desulphonated RNA is recovered, preferably, more than about 95% of the desulphonated RNA is recovered.

The method may further comprise processing or analysing the treated RNA sample.

The sample of nucleic acid to be treated may comprise RNA or a combination of both DNA and RNA. The sample may be purified or a crude extract.

The sample may be prepared or obtained from tissue, organ, cell, microorganism, biological sample, or environmental sample.

Preferably, the tissue or organ is selected from the group consisting of brain, colon, urogenital, lung, renal, hematopoietic, breast, thymus, testis, ovary, uterus, and mixtures thereof.

Preferably, the microorganism is selected from the group consisting of bacteria, virus, fungi, protozoan, viroid, and mixtures thereof.

Preferably, the biological sample is selected from the group consisting of blood, urine, faeces, semen, cerebrospinal fluid, lavage, saliva, swabs, cells or tissue from sources such as brain, colon, urogenital, lung, renal, hematopoietic, breast, thymus, testis, ovary, uterus, tissues from embryonic or extra-embryonic lineages, environmental samples, plants, microorganisms including bacteria, intracellular parasites, virus, fungi, protozoan, and viroid.

The method may be carried out in a reaction vessel. The reaction vessel can be selected from the group consisting of tube, plate, capillary tube, well, centrifuge tube, microfuge tube, slide, coverslip, and surface.

Advantageously, the method of the present invention may be carried out without causing substantial degradation or loss of the RNA sample and represents an improvement over commercially available kits.

Unlike known methods that use bisulphite to treat DNA, the present method results in substantially no degradation, or reduced degradation of the RNA, which means that the bisulphite treated RNA is suitable for downstream applications such as PCR as sufficient intact template remains. Thus the method according to the present invention can be used in situations where a precise measure of the amount of RNA present in a sample is required, such as gene expression analysis by qPCR and viral load monitoring of RNA viruses during drug therapy to determine the success of therapy.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of the invention.

In the context of the present invention, any one or more embodiments may be taken in combination with any other one or more embodiments and all such combinations are encompassed by the present disclosure.

In order that the present invention may be more clearly understood, preferred forms will be described with reference to the following drawings and examples.

### BRIEF DESCRIPTION OF THE DRAWINGS

FIG. 1 shows a comparison of recovery of bisulphite-treated RNA from RNA isolated from the prostate cancer cell line PC-3. RNA was isolated using Trizol™ (Invitrogen) according to the manufacturer's instruction. The RNA was then resuspended in the following desulphonation buffers. Tris/HCI buffer pH 9.5, 10.5, 11.5 and 12, then incubated at the specified temperatures and times. After incubation the RNA was precipitated using isopropanol and run on a precast (2%) agarose gel (Invitrogen). Standard DNA desulphonation was carried out at 95° C. for 30 minutes and as can be seen from FIG. 1, resulted in total degradation of the RNA sample rendering it useless for downstream applications.

FIG. 2 shows reverse transcriptase PCR (RT-PCR) carried out on bisulphite converted Acrometrix HCV samples using a high input of RNA and HIV-1 reverse transcriptase using various desulphonation times or no desulphonation to determine the shortest time of desulphonation that could be used to yield a positive RT-PCR signal. #1: 0 minutes desulphonation @ 76° C.; #2: 1 minute desulphonation @ 76° C.; #3: 2 minutes desulphonation @ 76° C.; #4: 5 minutes desulphonation @ 76° C.; #5: minutes desulphonation @ 76° C.; #6: 15 minutes desulphonation @ 76° C.; #7: minutes desulphonation @ 76° C. (control reverse transcriptase); #8: RT negative control; #9: PCR negative control.

FIG. 3 shows RT-PCR using several different reverse transcriptase enzymes using a low input of bisulphite converted Acrometrix HCV using various desulphonation times to determine the shortest time of desulphonation that could be used to yield a positive RT-PCR signal using different reverse transcriptase enzymes.

FIG. 4 shows RT-PCR on low input of bisulphite converted Acrometrix HCV RNA following a 5 minute desulphonation with TE buffer at different pH and temperatures to determine optimal conditions for desulphonation with this buffer.

FIG. 5 shows RT-PCR on varying low inputs of bisulphite converted Acrometrix HCV RNA following desulphonation with two buffers of different composition at 40° C. for 5 minutes. The results show that a range of buffers can be used to desulphonate at a broad range of pHs.

FIG. 6 shows the effect of pH, time and temperature on desulphonation of 10IU of bisulphite converted HCV RNA using 100 mM NaHCO₃, and demonstrates the broad range of conditions that can be tolerated by some buffers whilst maintaining the ability to effectively desulphonate.

FIG. 7 shows qPCR data obtained from PC3 RNA bisulphite treated for 5, 10, 15, 20, 25 and 30 minutes either in TE buffer pH 10.5 at 86° C. or pH 11.5 at 76° C.

FIG. 8 shows a dilution series of RNA treated with TE buffer pH 11.5 at 76° C. for 5 minutes then PCR amplified and the efficiency of the amplification reaction compared to unmodified wild type primers directed to the same region as the bisulphite treated primers.

FIG. 9 shows that magnetic beads can be effectively used to capture HCV RNA and allow the efficient bisulphite treatment, recovery, desulphonation and amplification of that RNA whilst still bound to the support.

FIG. 10 shows a comparison of bisulphite treatment of HCV RNA using the present invention and commercially available kits.

### DETAILED DESCRIPTION OF EMBODIMENTS OF THE INVENTION

Embodiments for treating RNA are described in non-limiting detail below.

The invention provides methods for the treatment and analysis of RNA samples. The methods are advantageous in that they provide a simple and highly efficient method for modification of RNA and can be used, for example, to examine the methylation pattern or changes in methylation of RNA, quantitation of gene expression and the ability to measure very low quantities of RNA for quantitation of viral copy number in response to drug therapy. The methods of the invention provide a simplified procedure with higher yields and higher molecular weight RNA without total destruction of the RNA, thus allowing the analysis of smaller amounts of RNA than would have previously been thought possible as well as easy application to a large number of samples.

The present invention relates to a method for bisulphite treating RNA, comprising:

reacting RNA with a bisulphite reagent at 50-90° C. for about 5-180 minutes so as to form treated RNA; and

recovering the treated RNA.

A preferred embodiment disclosed herein relates to a method for bisulphite treating RNA comprising:

optionally denaturing RNA to substantially remove any significant secondary structure present in the RNA;

reacting the RNA with a bisulphite reagent and incubating the reaction so as to form treated RNA;

reducing salt concentration to a level which will not substantially interfere with a nucleic acid precipitating step or binding of the treated RNA to a solid phase;

recovering the treated RNA; and

optionally carrying out partial or total desulphonation of the recovered treated RNA so as to remove sulphonate groups present on the treated RNA so as to obtain a treated RNA substantially free of sulphonate groups, or with a reduced number of sulphonate groups, without inducing significant amounts of RNA strand breakage.

The methods of the invention are particularly useful in the analysis of RNA samples.

The methods of the present invention are advantageous because they can be performed so that the nucleic acid sample, for example, strand/s of RNA are not broken or sheared to a significant extent. The methods of the invention also allow effective bisulphite treatment of very small quantities of RNA, such as 0.5 μg or less, eg, as little as about 0.2 μg or less, 0.1 μg or less, 500 ng or less, 250 ng or less, 100 ng or less, 50 ng or less, 15-150 attograms, or 60-120 attograms. (1 attogram=1×10⁻¹⁸ g)

The invention thus provides, in one embodiment, a method for treating RNA. In various embodiments of the invention the method may include some or all of the steps of denaturing RNA; incubating the RNA with a bisulphite reagent, thereby modifying nucleotides with sulphonate groups; diluting or otherwise purifying the bisulphite treated RNA from salts (including, bisulphite salts); precipitating the modified RNA or eluting the modified RNA from a solid phase; and reacting the modified RNA to partially or totally remove sulphonate groups.

The optional denaturing step can be performed, for example, by providing heat to the RNA. The optional desulphonation step is generally carried out under controlled conditions so as to partially or completely remove sulphonate groups present on the bisulphite treated RNA without substantially degrading the RNA.

The sample can be prepared from tissue, cells or can be any biological sample such as blood, urine, faeces, semen, saliva, swabs, cerebrospinal fluid, lavage, cells or tissue from sources such as brain, colon, urogenital, lung, renal, hematopoietic, breast, thymus, testis, ovary, uterus, tissues from embryonic or extra-embryonic lineages, environmental samples, plants, microorganisms including bacteria, intracellular parasites virus, fungi, protozoan, viroid and the like. The best described mammalian cell types suitable for treatment by the present invention are summarized in B. Alberts et al., 1989, The Molecular Biology of the Cell, 2^{nd} Edition, Garland Publishing Inc New York and London, pp 995-997.

The analysis of RNA from samples of human, animal, plant, bacterial, and viral origin is meant to cover all life cycle stages, in all cells, tissues and organs from fertilization until 48 hours post mortem, as well as samples that may be derived from histological sources, such as microscope slides, samples embedded in blocks, or samples extracted from synthetic or natural surfaces or from liquids.

The analyses also include RNA from prokaryotic or eukaryotic organisms and viruses (or combinations thereof), that are associated with human diseases in extracellular or intracellular modes.

Any suitable method for obtaining RNA material can be used. Examples include, but are not limited to, commercially available DNA, RNA kits or reagents, workstation, standard cell lysis buffers containing protease reagents and organic extraction procedures, which are well known to those of skill in the art.

The method according to the present invention may be carried out in a reaction vessel. The reaction vessel may be any suitable vessel such as tube, plate, capillary tube, well, centrifuge tube, microfuge tube, slide, coverslip or any suitable surface. The method is generally carried out in one reaction vessel in order to reduce the likelihood of degradation or loss of the nucleic acid sample.

Generally, the denaturation step comprises a heat treatment, however, other suitable denaturing agents could be used provided they do not affect the integrity of the initial RNA sample. It will be appreciated by those skilled in the art that in some circumstances this denaturation step may not be required as determined experimentally.

Generally, the bisulphite reagent is sodium bisulphite or sodium metabisulphite. Preferably, the bisulphite reagent is sodium metabisulphite. The bisulphite reagent causes sulphonation of cytosine bases to give cytosine sulphonate, which is followed by hydrolytic deamination of the cytosine sulphonate to uracil sulphonate. It will be appreciated, however, that any other suitable bisulphite reagent could be used (see Shapiro, R., DiFate, V., and Welcher, M, (1974) J. Am. Chem. Soc. 96: 906-912).

The incubation with the sulphonating reagent can be carried out at pH below 7 and at a temperature that favours the formation of the uracil sulphonate group. A pH below 7 is optimal for carrying out the sulphonation reaction, which converts the cytosine bases to cytosine sulphonate and subsequently to uracil sulphonate. However, the methods of the invention can be performed with the sulphonation reaction above pH 7, if desired.

The sulphonation reaction may be carried out in the presence of an additive capable of enhancing the bisulphite reaction. Examples of suitable additives include, but are not limited to DTT, quinol, urea, methoxyamine. Of these reagents, quinol is a reducing agent. Urea and methyoxyamine are agents added to improve the efficiency of the bisulphite reaction. It will be appreciated that other additives or agents can be provided to assist in the bisulphite reaction.

The sulphonation reaction results in methylated cytosines in the RNA sample remaining unchanged, while unmethylated cytosines are converted to uracils.

The sulphonation reaction is generally carried out at a temperature of about 50-90° C. for about 5-180 minutes. In various preferred embodiments the sulphonation reaction is carried out at a temperature of about 50-90° C. for about 5-150 minutes, about 50-90° C. for about 5-120 minutes, about 50-90° C. for about 5-90 minutes, or about 50-90° C. for about 5-60 minutes. In alternative embodiments, the sulphonation reaction is carried out at about 50-90° C. for a time sufficient to achieve a desired level of sulphonation, for example, about 5 minutes, 10 minutes, 15 minutes, 20 minutes, 25 minutes, 30 minutes, 35 minutes, 40 minutes, 45 minutes, 50 minutes, 55 minutes, 60 minutes, 70 minutes, 80 minutes, or about 90 minutes. In a particularly preferred embodiment, the sulphonation reaction is carried out at about 70° C. for about 20 minutes.

Typically, the concentration of bisulphite reagent is from about 1 M to about 6 M, preferably from about 2 M to about 4 M, more preferably about 3 M.

Reaction conditions found to work particularly well in various embodiments of the invention are as follows. The RNA, or other nucleic acids, to be treated is made up to a volume of 20 μl. Then 208 μl of a freshly prepared solution of 3 M sodium metabisulphite (BDH AnalaR #10356.4D) pH 5.0 (the pH may be adjusted by the addition of 10 M sodium hydroxide (BDH AnalaR #10252.4X) along with 12 μl of a 100 mM quinol solution (BDH AnalaR #103122E). The concentration of quinol added can be anything in the range of about 10 to 500 mM as determined experimentally. The solution is then mixed well and optionally overlayed with 208 μl of mineral oil (Sigma molecular biology grade M-5904) or performed in a 0.2 ml tube in a heated lid thermocycler. The sample is then left for 10 minutes to about 3 hours, preferably 20 minutes, at a suitable temperature, for example, 60-90° C., preferably 70° C., or another suitable temperature, to allow time for full bisulphite conversion. This reaction step may also be carried out whilst the RNA is attached to a solid phase. It will be understood by those skilled in the art that the volumes, concentrations and incubation times and temperatures described above can be varied so long as the reaction conditions are suitable for sulphonation of the nucleic acids, eg, RNA.

The method may include a dilution step so that the salts inhibitory to subsequent reactions are not co-precipitated with the sulphonated nucleic acids, ie sulphonated RNA. Preferably, the salt concentration is diluted to less than about 1 M, eg, less than about 0.5M. Generally, the dilution step is carried out using water or buffer to reduce the salt concentration to below about 1 M, eg, below about 0.5M. For example, the salt concentration is generally diluted to less than about 1 mM to about 1M, in particular, less than about 0.5M, less than about 0.4M, less than about 0.3M, less than about 0.2M, less than about 0.1 M, less than about 50 mM, less than about 20 mM, less than about 10 mM, or even less than about 1 mM, if desired. One skilled in the art can readily determine a suitable dilution that diminishes salt precipitation with the nucleic acids so that subsequent steps can be performed with minimal further clean up or manipulation of the nucleic acid sample. The dilution is generally carried out in water but can be carried out in any suitable buffer, for example Tris/EDTA or other biological buffers, so long as the buffer does not precipitate significantly or cause the salt to precipitate significantly, with the nucleic acids so as to inhibit subsequent reactions. Generally, precipitation is carried out using a precipitating agent such as an alcohol. An exemplary alcohol for precipitation of nucleic acids can be selected from isopropanol, ethanol or any other suitable alcohol.

In alternative embodiments, a binding reagent can be added to the sample to facilitate the binding of the reacted RNA to a solid phase support for subsequent purification steps. The bound RNA can then be washed to remove salts (eg bisulphite salts) and any other unwanted impurities, then eluted from the solid support into an appropriate elution buffer. This would particularly suit a fixed solid support, such as a column, or magnetic movable solid support, such as coated magnetic beads.

In accordance with embodiments of the present invention, one or more steps may be performed on a solid support. In one embodiment, all steps are performed on a solid support. In a preferred embodiment, the desulphonation step is performed on a solid support.

The optional desulphonation step may be carried out by adjusting the pH of the precipitated treated RNA up to a maximum pH of about 11.5. However in some embodiments a lower pH may be preferred to minimise RNA degradation. Exposure to highly alkaline environments, eg, pH 12 or greater, can result in total degradation of RNA molecules and therefore, exposure to the alkaline pH treatment is minimized to avoid or limit strand breaks. The desulphonation step can be carried out efficiently at around pH 7.0 to 11.5 with a suitable buffer or alkali reagent. Examples of suitable buffers or alkali reagents include buffers having a pH 7.0-11.5, such as, but not limited to, TE ('Tris-EDTA'), CAPS ('N-cyclohexyl-3-aminopropanesulfonic acid'), phosphate, glycine, methylamine and sodium hydrogen carbonate. In preferred embodiments, the desulphonation may be carried out at a pH between 8.5 and 11.5. In particularly preferred embodiments, the desulphonation may be carried out at pH 8.7, 10.5 or pH 11.5. Particularly preferred buffers include TE, sodium bicarbonate and CAPS. It will be appreciated by persons skilled in the art that suitable buffers or alkali reagents can be selected from the vast range of known buffers and alkali reagents available.

Generally, temperature ranges for the desulphonation step are 0° C., or less, up to about 90° C. and treatment times can vary from about 1 minute to about 30 minutes, or longer (eg, up to about 45 or 60 minutes) depending on the conditions used. In preferred embodiments of the present invention, the desulphonation is carried out at a temperature of about 30-50° C. for about 1-30 minutes. In a particularly preferred embodiment the desulphonation is carried out at about 40° C. for about 2-20 minutes, more preferably about 5-10 minutes, more preferably about 5 minutes. One skilled in the art can readily determine a suitable time and temperature for carrying out the desulphonation reaction. Temperatures below room temperature can also be used so long as the incubation time is increased to allow sufficient desulphonation. Thus, the desulphonation step may be carried out at less than 10° C., about 5° C., about 10° C., about 20° C., about 22° C., about 25° C., about 30° C., about 35° C., about 37° C., about 40° C., about 45° C., about 50° C., about 55° C., about 60° C., about 65° C., about 70° C., about 75° C., about 76° C., about 80° C., about 85° C., about 86° C., about 90° C. A particularly useful temperature for carrying out the desulphonation reaction is about 40-75° C., preferably about 40° C.

In some embodiments, for example, when using reverse transcriptases that are capable of copying sulphonated RNA, it may not be necessary to desulphonate the nucleic acid at all. Whether or not desulphonation is required or desired can easily be determined experimentally by those skilled in the art.

Another advantage of the present invention is that the method may be carried out in a much shorter time frame than treatment methods using other commercially available kits, such as the methyl SEQr bisulphite conversion kit (Applied Biosystems, cat #4374960), the Methylamp-96 DNA modification kit (Epigentek, cat #P-1008), the EpiTect bisulphite kit (Qiagen, cat #59104), and the EZ DNA methylation direct kit (Zymo Research, cat #D5020).

The present invention provides methods for the efficient characterisation of RNA. The methods allow efficient sulphonation and desulphonation steps to be carried out on the RNA sample. However, it is understood that neither of the sulphonation nor desulphonation steps need be carried out to completion, only sufficiently to subsequently characterize the presence or absence of the nucleic acid, as disclosed herein. One skilled in the art can readily determine whether these steps should be carried out to near completion or whether incomplete reactions are sufficient for a desired analysis. For example, when a small number of cells or a small amount of RNA is used, it is generally desired that a more complete reaction be performed. When larger quantities of nucleic acid sample are being characterised, a less complete reaction can be carried out while still providing sufficient reaction products for subsequent analysis of the RNA sample. A particular advantage of the present invention is that it allows very small amounts of RNA to be treated and characterised, for example, amounts of about 0.5 μg or less, eg, about 0.2 μg or less, about 0.1 μg or less, 500 ng or less, 250 ng or less, 100 ng or less, about 15-150 attograms, or about 60-120 attograms.

As disclosed herein, the invention provides methods for conveniently treating RNA. The methods can be used for the analysis of the methylation state of a RNA molecule, or a method for gene expression analysis or for the monitoring of low levels of an RNA virus such as Hepatitis C (HCV) for viral load monitoring in response to drug treatment.

An advantage of the present invention is that the desalting step is carried out in a highly efficient manner by diluting the salt concentration and precipitating the nucleic acids or binding the nucleic acids to a solid support. The dilution step reduces the salt concentration below an amount that, when the nucleic acid is precipitated or bound to the solid support, does not interfere with subsequent steps, such as desulphonation. The precipitation step is highly efficient and can optionally include carriers that increase the efficiency of nucleic acid precipitation. The use of solid supports such as columns or magnetic beads allows for optimal recovery, reduced time to results and is readily automatable. Thus, the methods of the invention minimize loss and increase recovery of nucleic acid samples. Accordingly, the methods of the invention provide the additional advantage of allowing very small amounts of starting material to be used and efficiently characterised with respect to methylation, gene expression and detection of pathogens.

Further, when the method includes a desulphonation step the use of a buffer solution at slightly alkaline pH can be used to decrease the likelihood that the RNA of interest becomes substantially fragmented. Increasing the pH of the buffered solution to much above pH 11.5 may lead to very substantial fragmentation of high molecular weight nucleic acids especially RNA. Therefore, when it is desired to minimize such fragmentation, an alkaline pH below about pH 11.5, eg, from about 8.5 to 11.5 is generally used.

Yet another advantage of the invention is that the reactions can be carried out in a single tube or vessel for each sample, thus minimizing sample loss and allowing the processing of numerous samples. A further advantage of the method of the invention compared to previous methods is that the RNA, once sulphonated, can be resuspended in a buffer having a basic pH to carry out the desulphonation step rather than requiring the addition of strong base which would completely destroy the target RNA, as in the method described by Clark et al., 1994.

The methods of the invention can be used to characterise the methylation state of a RNA species whether mRNA, tRNA, rRNA, microRNA, shRNA, siRNA or any other species of RNA of interest, tissue or organism. The methods of the invention can also be used in conjunction with genomic sequencing methods such as those described by Frommer et al., Proc. Natl. Acad. Sci. USA 89:1827-1831 (1992), which is incorporated herein by reference.

The invention additionally provides a method of determining the methylation state of a sample, or to quantify the gene expression profiles of a sample or quantitate the circulating levels of a virus in a patient sample. The method can be carried out on a sample using the method of the invention for treatment of RNA. The method for determining the methylation state of a sample can be carried out in parallel with a test sample and a control sample so that the methylation state of the sample can be compared and determined relative to a reference sample; again this can be applied to gene expression or viral load monitoring assays. For example, the samples can be compared to determine whether there is an increase or decrease of methylation in general or at particular sites. Such a determination can be used to diagnose and/or determine the prognosis of a disease, as discussed herein. The method can further include reporting of the methylation state of a sample, for example, in a diagnostic application such as the presence and or quantitation of an RNA based virus such as HCV or HIV.

It is understood that the components of the method of the invention can be provided in the form of a kit. The kit can contain appropriate chemical reagents, reaction vessels, eg, tubes and instructions for carrying out the method of the invention.

### EXAMPLES

### Methods and reagents

Chemicals were obtained as follows: Agarose from BioRad (Hercules Calif.; certified molecular biology grade #161-3101); Acetic acid, glacial, from BDH (Kylsyth, Australia; AnalaR 100015N); ethylenediamine tetraacetic acid (EDTA) from BDH (AnalaR 10093.5V); Ethanol from Aldrich (St. Louis Mo.; 200 proof E702-3); Isopropanol from Sigma (St. Louis Mo.; 99%+ Sigma 1-9516); Mineral oil from Sigma (M-5904); Sodium acetate solution 3M from Sigma (S-7899); Sodium chloride from Sigma (ACS reagent S9888); and Sodium hydroxide from BDH (AnalaR #10252.4X).

Enzymes/Reagents were obtained as follows: PCR master mix from Promega (Madison Wis.; #M7505); Superscript III reverse transcriptase (Invitrogen); HIV reverse transcriptase (Ambion #AM2045); iScript reverse transcriptase kit (Biorad #1708897) and DNA markers from Sigma (Direct load PCR low ladder 100-1000 bp, Sigma D-3687 and 100-10 Kb, Sigma D-7058).

Solutions were as follows: (1) 10 mM Tris/0.1 M EDTA, pH 7.0-12.5; (2) 3 M Metabisulphite (5.6 g in 10 ml water with 500 μl 10 N NaOH (BDH AnalaR #10356.4D); (3) 100 mM Quinol (0.55 g in 50 ml water; BDH AnalaR #103122E); (4) 100 mM NaHCO₃, pH 8.0-11.5 (BDH #10247); (5) 10 mM CAPS, pH 11-11.5 (Sigma #C6070); (6) 50×TAE gel electrophoresis buffer (242 g Trizma base, 57.1 ml glacial acetic acid, 37.2 g EDTA and water to 1 l); and (7) 5× Agarose gel loading buffer (1 ml 1% Bromophenol blue (Sigma B6131), 1 ml Xylene Cyanol (Sigma X-4126), 3.2 ml Glycerol (Sigma G6279), 8 μl 0.5 M EDTA pH 8.0, 200 μl 50×TAE buffer and water to 10 ml).

### Tissues and Cell Lines

RNA was isolated from the prostate cancer cell line PC3 using Trizol™ (Invitrogen) as instructed in the manufacturer's datasheet.

HCV RNA was isolated OptiQual® HCV RNA high positive control (Acrometrix cat #96-0203) using the QiaAmp UltraSens (Qiagen) viral kit according to the manufacturer's instructions and resuspended at a final concentration of 5,000 IU/μl.

### Bisulphite Conversion of RNA

3.35 g Sodium bisulphite (Sigma 59000 500 g; lot number 116K0761) was dissolved in 5 ml Xceed reagent 1 (Methyleasy Xceed kit, Human Genetic Signatures, Sydney, Australia). The reagent was heated at 80° C. until fully dissolved then allowed to cool.

0.11 g of Hydroquinone (Merck 8.22333.0250; lot number K36100033 702) was dissolved in 10 ml nuclease-free water.

5 μl of RNA was mixed with 220 μl bisulphite reagent and 12 μl quinol in a PCR tube and incubated at 70° C. for 20 minutes in a PCR machine.

800 μl nuclease-free water was added along with 2 μl glycoblue (Ambion AM9515; lot number 0705003), the sample mixed well, then 1 ml isopropanol was added and the samples incubated at 4° C. for 1 hour.

The RNA was pelleted by centrifuging at 16 000×g for 20 minutes at 4° C. The supernatant was discarded and the pellet washed with 1 ml 70% ethanol with moderate vortexing. The sample was recentrifuged at 16 000×g for 7 minutes at 4° C.

The supernatant was discarded and the pellet air dried for a few minutes.

The pellet was resuspended in 70 μl desulphonation buffer (Xceed reagent 5, Methyleasy Xceed kit, Human Genetic Signatures, Sydney, Australia) and desulphonated at 76° C. for 0-15 minutes in a PCR machine.

The RNA was cooled, then 11 μl RNA was added to 2 μl of a mastermix comprising the following per reaction:

1 μl 10 mM dNTPS

1 μl random H primers (300 ng/μl)

The sample was heated at 65° C. for 5 minutes, then placed on ice for at least 1 minute, before adding 7 μl of a mastermix comprising the following per reaction:

| HIV-RT | Control RT |
|---|---|
| 2 μl 10x HIV-RT Buffer | 4 μl 5x FS buffer |
| 1 μl HIV RT (1 U/μl) | 1 μl 0.1 M DTT |
| 1 μl Rnase OUT | 1 μl Superscript III |
| 3 μl water | 1 μl Rnase OUT |

The samples were mixed and the reverse transcription carried out as follows; 25° C. for 2 mins, 27° C. for 2 mins, 29° C. for 2 mins, 31° C. for 2 mins, 33° C. for 2 mins, 35° C. for 2 mins, 37° C. for 30 mins, 45° C. for 10 mins, 50° C. for 10 mins, 70° C. for 5 mins, then soaked at 15° C.

5 μl of the cDNA was taken for analysis by PCR, comprising the following per reaction:
37.5 μl Promega mastermix
1.0 μl F1 primer (100 ng/μl)
1.0 μl R0 primer (100 ng/μl)
5.5 μl water

Cycling conditions were as follows:

| | | |
|---|---|---|
| 95° C., 3 mins | | |
| 95° C., 10 secs | | |
| 53° C., 1 min | 40x | {close oversize brace} |
| 68° C., 1 min | | |
| 68° C., 7 mins | | |

The following equipment was used: the PCR machine was a ThermalHybaid PX2 (Sydney, Australia) the Gel Documentation System was a Kodak UVItec EDAS 290 (Rochester N.Y.), and the microfuge was an Eppendorf 5415-D (Brinkman Instruments; Westbury N.Y.).

### RNA Separation

2% Agarose pre-cast gels (Invitrogen) were used. The RNA sample of interest or RT-PCR products were directly loaded into the wells of the plate and the gel resolved using the mother-base (Invitrogen).

### Bisulphite Treatment of RNA

An exemplary protocol demonstrating the effectiveness of the bisulphite treatment according to the present invention is set out below. The protocol successfully resulted in recovering substantially all RNA treated. It will be appreciated that the volumes or amounts of sample or reagents can be varied.

RNA, in a final volume of 20 μl, was heated at 80° C. for 2 minutes to denature any secondary structure present within the target molecule. Incubation at temperatures above 50° C. can be used to improve the efficiency of denaturation of secondary structure. Molecules that are suspected of having a high degree of secondary structure may require higher temperatures to remove all structure prior to bisulphite treatment. In some cases, it may not be necessary to heat denature the RNA prior to treatment. RNA denaturation can be performed at any temperature from about 37° C. to about 90° C. and can vary in length from about 5 minutes to about 8 hours.

After the incubation, 208 μl 3 M sodium metabisulphite (5.6 g in 10 ml water with 500 μl 10 N NaOH; BDH AnalaR #10356.4D; freshly made) and 12 μl of 100 mM Quinol (0.55 g in 50 ml water, BDH AnaIR #103122E; freshly made) were added in succession. Quinol is a reducing agent and helps to reduce oxidation of the reagents. Other reducing agents can also be used, for example, dithiothreitol (DTT) is especially useful, as it is known to inhibit the action of RNases. The sample was overlaid with 200 μl of mineral oil or the reaction performed in a 0.2 ml PCR tube in a heated lid thermocycler. The overlaying of mineral oil prevents evaporation and oxidation of the reagents but is not essential. The sample was then incubated for 20 minutes at 70° C. Generally, bisulphite treatment may be performed at any temperature from about 50° C. to about 90° C. and can vary in length from about 5 minutes to about 180 minutes.

After the treatment with sodium metabisulphite, the oil was removed, and 1 μl tRNA (20 mg/ml) or 2 μl glycoblue were added if the RNA concentration was low or to help visualise the pellet after precipitation. These additives are optional and can be used to improve the yield of RNA obtained by co-precipitating with the target RNA especially when the RNA is present at low concentrations. The use of additives as carrier for more efficient precipitation of nucleic acids is generally desired when the amount of nucleic acid is <0.5 μg.

An isopropanol cleanup treatment was performed as follows: 800 μl of water were added to the sample, mixed and then 1 ml isopropanol was added. The water or buffer reduces the concentration of the bisulphite salt in the reaction vessel to a level at which the salt will not precipitate along with the target nucleic acid of interest. The dilution is generally about ¼ to 1/1000 so long as the salt concentration is diluted below a desired range, as disclosed herein.

The sample was mixed again and left at 4° C. for a minimum of 5 minutes. The precipitation step can be left for any period of time from 5 minutes to several hours, but preferably the sample will be allowed to precipitate for 1 hour or less. The sample was spun in a microfuge for 10-15 minutes and the pellet was washed 1×-2× with 70-80% ethanol, vortexing each time. This washing treatment removes any residual salts that precipitated with the nucleic acids.

The pellet was allowed to dry and then resuspended in a suitable volume of TE (10 mM Tris/0.1 mM EDTA), or other buffer, pH 7.0-11.5 such as 50 μl. Buffer at pH 11.5 has been found to be particularly effective for TE, although lower pHs can be effectively used with other buffers. The sample was incubated at 20° C. to 90° C. for about 1-30 minutes, as needed to suspend and desulphonate the nucleic acids.

### Analysis of PC3 Cells and Sensitivity of PCR Amplification

Cultures of PC3 cells were grown under standard conditions to 90% confluence. Cells were trypsinised, washed, then counted using a haemocytometer. Cells were then diluted as required. The cells were lysed using Trizol (Invitrogen) as described by the manufacturer's instructions and the RNA then modified using the sulphonation methods described above.

The RNA was then diluted in 0.2 ml PCR tubes as follows; 1/100, 1/1000, 1/10000 and 1/100000 in 10 μl of T/E pH 8.0. RNA was amplified for 40 cycles as previously stated.

### Effect of pH, Time and Temperature on Degradation of Bisulphite Treated RNA

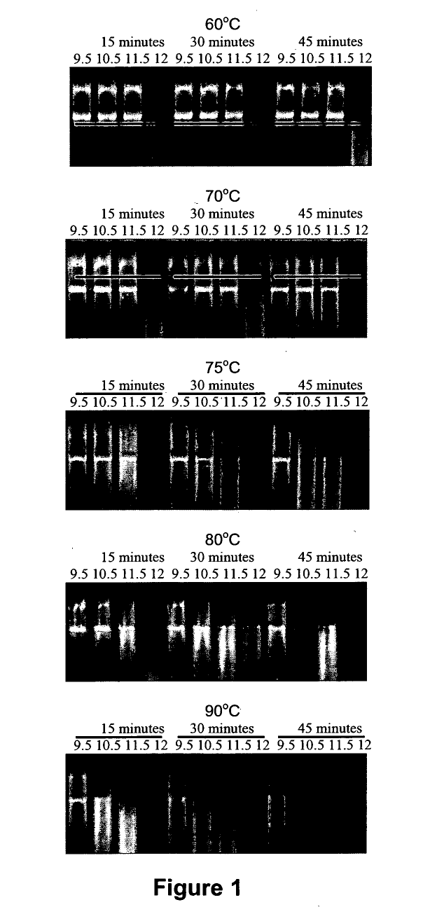

As can be seen from FIG. 1, the higher the temperature and pH the more likely that the RNA will be degraded. The normal desulphonation temperature for bisulphite treated DNA is 95° C. for 30 minutes and as can be seen from FIG. 1, even treatment at 90° C. for 15 minutes results in total degradation of the RNA making the material useless for downstream applications such as PCR. In order to produce accurate quantitation of RNA for applications such as viral load monitoring significant degradation of the starting material must be avoided. Degradation of the RNA would be random in nature therefore this randomness would impact on sample to sample variability and lead to an inaccurate estimation of viral load which could have serious consequences on patient management regimes. As can be seen from FIG. 1 even at 70° C. significant degradation of the RNA can be observed after 15 minutes incubation indicating that the desulphonation time should be reduced at this temperature to minimise damage to the RNA. At 60° C. virtually no degradation can be observed, except when using the high pH desulphonation buffer (pH 12), of any sample over the entire time course experiment. Thus from these results it might suggest that the optimal time/pH and temperature range when using TE as a desulphonation buffer would be around 60° C. to 70° C. or less using a pH of 11.5 or less for less than 15 minutes.

### Effect of Time and Temperature on RT-PCR Amplification of Bisulphite Treated RNA Using HIV RT

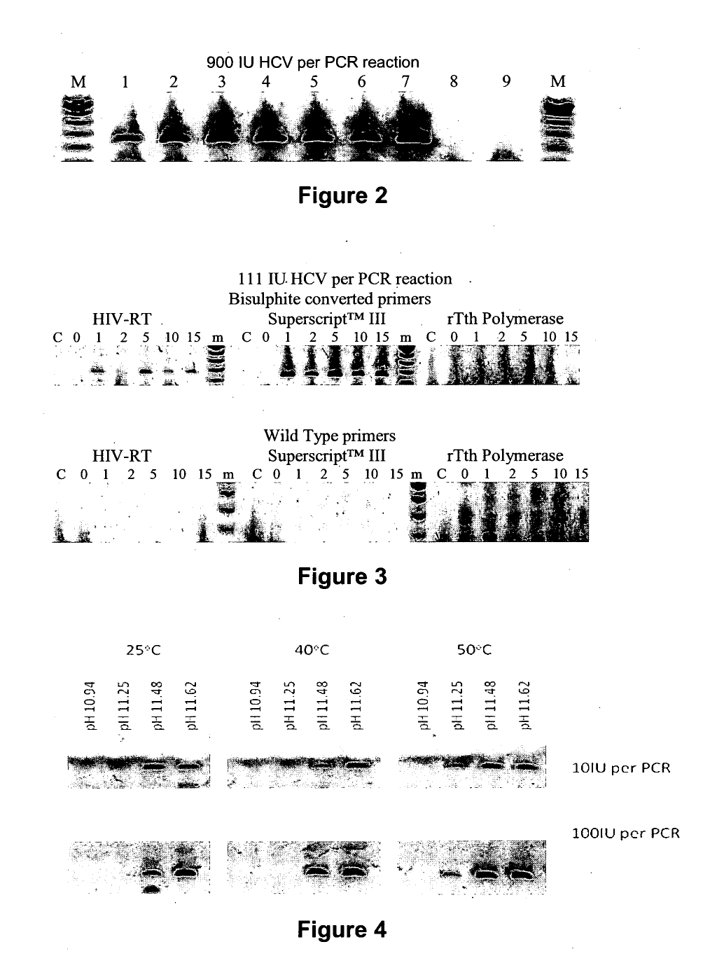

FIG. 2 and FIG. 3 show that it is possible to amplify bisulphite treated RNA without any significant desulphonation as long as there is a relatively large amount of starting material using HIV RT. However, when the amount of starting material is reduced then at least 1-minute desulphonation is required to produce optimal signals in this example.

Remarkably it also seems from the data (see FIG. 2 and FIG. 3) that certain RNA reverse transcriptase (RT) enzymes appear to be a lot more “promiscuous” than their DNA polymerase counterparts, as to amplify bisulphite treated DNA for a desulphonation time of at least 20 minutes or more at 80° C. is commonly required to generate any amplified bisulphite treated DNA. However, with the reverse transcriptase enzymes, the RNA can be amplified without any desulphonation at all as long as the concentration of target is fairly high (see FIG. 2 and FIG. 3). This indicates that the reverse transcriptase enzymes have the ability to bypass bulky lesions on the RNA molecule such as sulphate groups whereas sulphate groups on the DNA apparently form a blockage that stop the polymerase from copying the treated DNA when the lesion is reached. Thus, this hereto-unknown property of RT enzymes is extremely advantageous when it comes to copying a DNA strand from bisulphite treated RNA as it raises the possibility that the desulphonation step may be completely avoided in some cases. As can been seen from FIG. 1 it is the desulphonation step that causes the most damage to the bisulphite treated RNA due to the high pH and temperatures required for removal of the sulphate groups, thus removal of this step not only reduces the time taken but will greatly assist in reducing or preventing RNA degradation.

### Effect of Time, Temperature and pH of Desulphonation Buffers on RT-PCR Amplification of Bisulphite Treated RNA

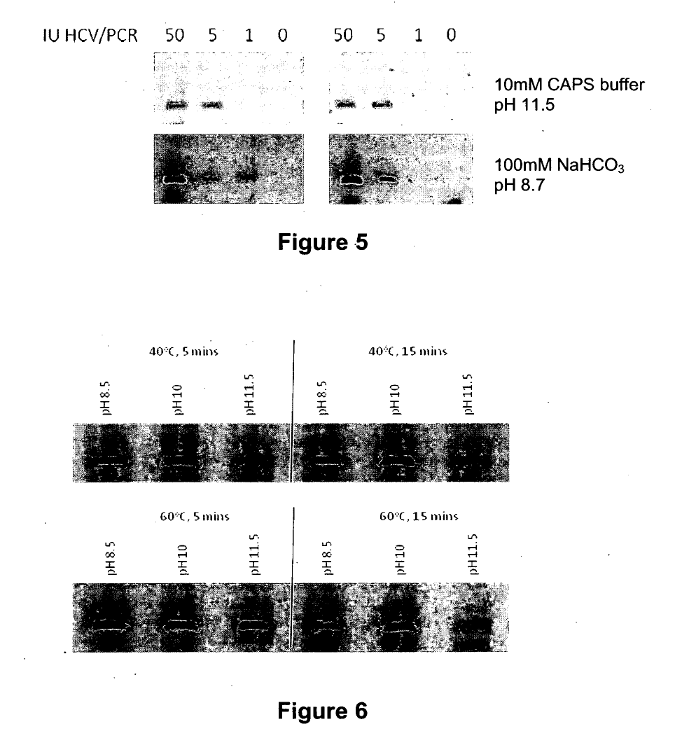

FIGS. 4, 5 and 6 demonstrate that the effective desulphonation of bisulphite converted RNA samples depends not only on the temperature, time and pH of the buffer, but also on the composition of the buffer. Thus for different buffers, the optimal pH for desulphonation of identical samples varies between pH 8.7 and 11.5, for NaHCO₃ and CAPS, respectively, when desulphonation is carried out for 5 minutes at 40° C. (FIG. 5). For TE buffer, increasing the temperature results in effective desulphonation using a lower pH (FIG. 4), whereas 100 mM NaHCO₃ can effectively be used to desulphonate RNA over a wide range of pHs (FIG. 6).

A particular advantage of this invention is the demonstration of amplification of exceedingly low amounts of bisulphite treated RNA, such as 1-10IU of HCV RNA (equivalent to approximately 15-150 attograms of RNA), which thus confirms that the RNA is not significantly degraded. Bisulphite treatment and subsequent detection of RNA has never before been demonstrated anywhere near to these low levels. Hence, it is possible to use a range of buffers at different pH and desulphonate at varying times and temperatures, depending on the RNA sample and desired experimental design.

### Real-Time PCR Analysis of Desulphonation Reaction Conditions Using RNA

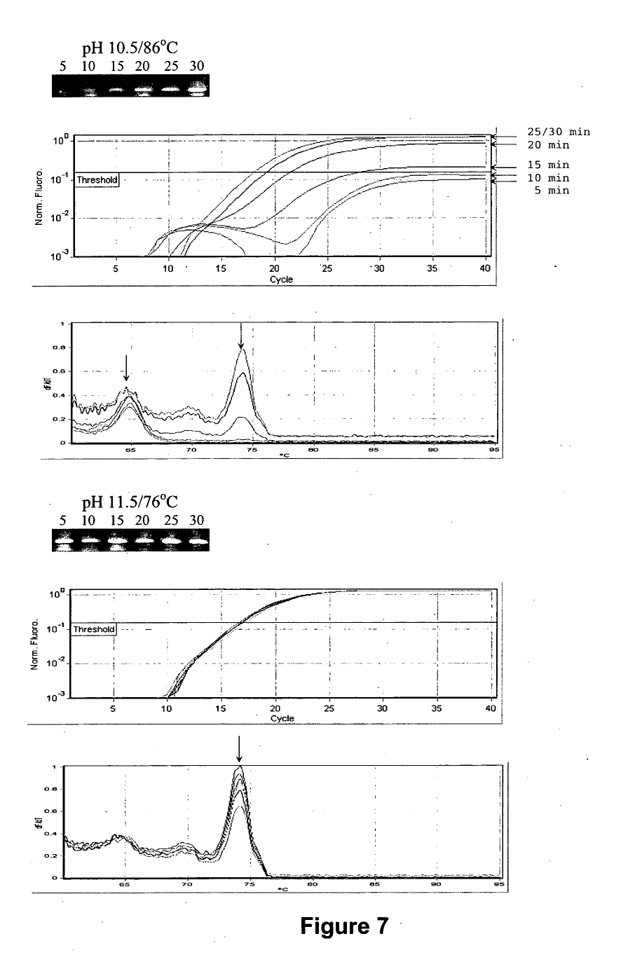

FIG. 7 shows that at reduced pH (10.5) even at high temperature (86° C.) optimal amplification curves are only generated after at least 20 minutes of desulphonation with TE buffer prior to reverse transcription and PCR. However, when the pH is increased to 11.5 and the temperature decreased this actually improves the amplification efficiency of the cDNA dramatically with no apparent difference in amplification after only 5 minutes desulphonation.

The results show bisulphite treatment of RNA using a single round one-step Reverse-Transcriptase PCR. Reverse transcription was carried out using a gene specific primer followed by 40 cycles of PCR amplification. Fluorescence was measured by the incorporation of Syto 9 into the reaction. It can be seen that using pH 11.5/76° C. desulphonation can be carried out in as little as 5 minutes. This is surprising as it would have been thought from the prior art that the increased pH would be detrimental to the RNA. The results from this experiment indicate that the actual desulphonation of the RNA occurs very rapidly under optimised conditions.

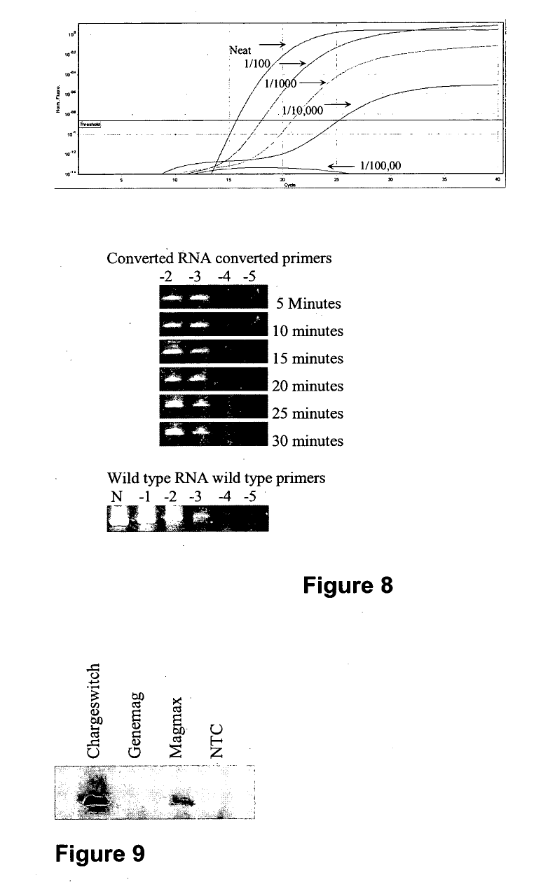

FIG. 8 indicates that with optimal conditions for bisulphite treatment and desulphonation the amplification of bisulphite treated RNA produces amplification signals that are equivalent to wild type RNA indicating that the improved bisulphite procedure results in virtually no loss of input RNA.

The results in FIG. 8 show bisulphite treatment of RNA using a single round one-step Reverse-Transcriptase PCR using a dilution series of input RNA. Reverse transcription was carried out using a gene specific primer followed by 40 cycles of PCR amplification. Fluorescence was measured by the incorporation of Syto 9 into the reaction. The results show that the reaction does not suffer any loss of sensitivity when the desulphonation time is reduced to only 5 minutes. In addition, dilution studies show that bisulphite treatment of the RNA does not result in any significant loss of sensitivity when compared to untreated RNA.

### Use of Solid Supports During the Bisulphite Treatment

FIG. 9 shows that different solid supports can be used during the bisulphite treatment of RNA. Acrometrix HCV RNA was bound to magnetic beads from three different suppliers (Chargeswitch, Invitrogen; Genemag, Chemicell; and Magmax, Ambion), bisulphite treated whilst bound to the beads, washed to remove excess bisulphite reagent and then eluted and desulphonated in a single step prior to reverse transcription with iScript reverse transcriptase (Biorad) and subsequent PCR amplification. Two of the three bead types worked in this example and are therefore compatible with the bisulphite treatment and retain their RNA binding capability. Other compatible supports such as columns may also be used during this procedure. The use of a solid phase allows for automation of the procedure and/or reduced time to results compared with precipitation-based methods. The treated RNA may be bound to the solid phase at any point during the procedure as required or throughout the entire procedure, including PCR amplification, if desired.

Comparison with Commercially Available Bisulphite Kits

A comparison of bisulphite conversion of HCV RNA was made using four leading commercially available bisulphite conversion kits and the results compared to the inventive method (referred to as the ‘HGS’ method).

Acrometrix HCV RNA was purified and 10,000 copies, 5,000 copies, 1,000 copies, 500 copies, 100 copies, 50 copies, 20 copies and 0 copies were bisulphite converted using either the HGS method as disclosed herein, the methyl SEQr bisulphite conversion kit (Applied Biosystems, cat #4374960), the Methylamp-96 DNA modification kit (Epigentek, cat #P-1008), the EpiTect bisulphite kit (Qiagen, cat #59104) or the EZ DNA methylation direct kit (Zymo Research, cat #D5020), according to the manufacturer's instructions (Table 1).

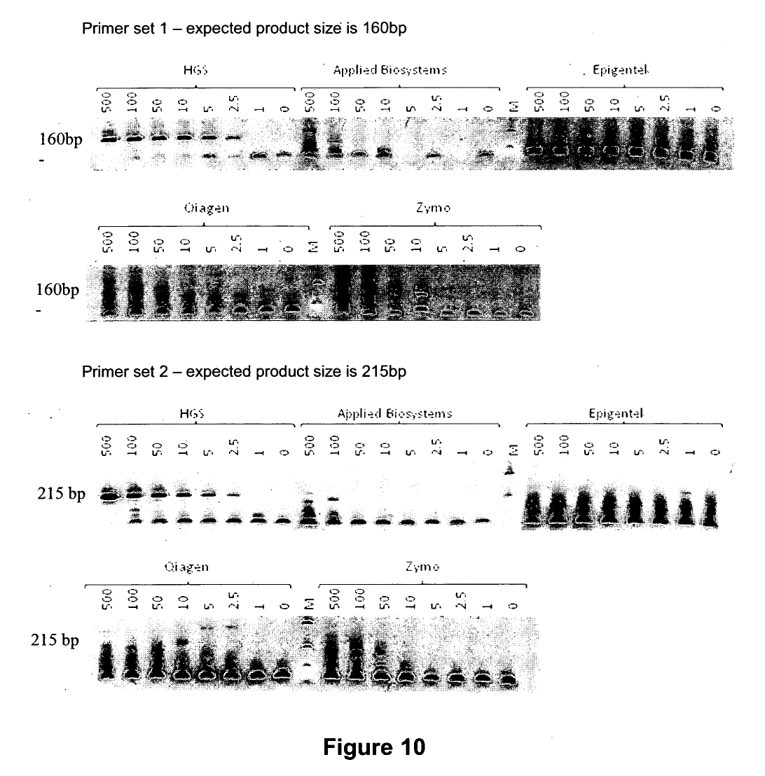

Following desulphonation and/or elution, the RNA was reverse transcribed as disclosed herein. 1 μl out of the 20 μl total cDNA was PCR amplified using two different primer sets which are designed to specifically amplify bisulphite converted HCV. Thus, in the PCR, there is 500, 100, 50, 10, 5, 2.5, 1, and 0 copies HCV/reaction. As is evident from FIG. 10, the HGS method was the only method to effectively retain all the HCV or allow for efficient amplification of the HCV.

TABLE 1
Bisulphite conversion and desulphonation conditions

| | Temp/Time | | | | |
|---|---|---|---|---|---|
| | Bisulphite | Temp/Time | | Total | Retention |
| Method | treatment | Desulphonation | Notes | time | of RNA |
| Applied | 50° C., 16 hrs | Room temp, | Clean up and | 18.5 hr | No |
| Biosystems | | 5 min | desulphonation | | |
| | | | performed on | | |
| | | | column | | |
| Epigentek | 98° C., 6 min | Room temp, | Clean up and | 3 hr | No |
| | 65° C., 90 min | 10 min | desulphonation | | |
| | | | performed on filter | | |
| | | | plate | | |
| Qiagen | 99° C., 5 min; | Room temp, | Clean up and | 6 hr | No |
| | 60° C., 25 min | 15 min | desulphonation | | |
| | 99° C., 5 min | | performed on spin | | |
| | 60° C., 85 min | | column | | |
| | 99° C., 5 min | | | | |
| | 60° C., 175 min | | | | |
| Zymo | 98° C., 8 min | Room temp, | Clean up and | 4.5 hr | No |
| | 64° C., 3.5 hrs | 20 min | desulphonation | | |
| | | | performed on | | |
| | | | column | | |
| HGS* | 70° C., 20 min | 40° C., 5 min | Precipitation | 2-2.5 hr | Yes |
| | | | based clean up | | |

### Summary

With bisulphite treated DNA, the samples are often desulphonated for at least 20 minutes or more at a temperature of at least 80° C. in order to generate any significant amplification. However, this is in contrast with various reverse transcriptase enzymes with which the RNA can be amplified without any desulphonation at all as long as the concentration of target is fairly high (see FIG. 2 and FIG. 3), or with as little as 5 minutes desulphonation with lower starting RNA concentrations (FIGS. 4, 5 and 6). The cDNA generated can then be further amplified by PCR using traditional methods without any subsequent treatment. From these results it would seem that some reverse transcriptase enzymes are much more efficient at amplifying bisulphite treated nucleic acids than their DNA polymerase counterparts. This, and the optimized conversion and desulphonation conditions, means that bisulphite conversion of RNA can be effectively carried out, thus resulting in significant improvement in the ability of the method to keep the RNA molecules intact during the conversion process leading to better sensitivity of any RNA based assay.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for bisulphite treating RNA, comprising:
reacting RNA with a bisulphite reagent at 50-90° C. for 5-180 minutes so as to form treated RNA; and
recovering the treated RNA.

2. The method according to claim 1, comprising reacting RNA with a bisulphite reagent at 50-90° C. for 5-120 minutes.

3. The method according to claim 2, comprising reacting RNA with a bisulphite reagent at 50-90° C. for 5-90 minutes.

4. The method according to claim 3, comprising reacting RNA with a bisulphite reagent at 50-90° C. for 5-60 minutes.

5. The method according to claim 4, comprising reacting RNA with a bisulphite reagent at 70° C. for 20 minutes.

6. The method according to any one of claims 1 to 5 further comprising carrying out partial or total desulphonation of the recovered RNA.

7. The method according to claim 6, wherein desulphonation is carried out at an alkaline pH up to about pH 11.5.

8. The method according to claim 6 or 7, wherein desulphonation is carried out at a temperature of 0-90° C. for 1-30 minutes.

9. The method according to claim 8, wherein desulphonation is carried out at a temperature of 20-50° C., for 5-20 minutes.

10. The method according to any one of claims 1 to 9, wherein more than 70% of the treated RNA is recovered.

11. The method according to claim 10, wherein more than 80% of the treated RNA is recovered.

12. The method according to claim 11, wherein more than 90% of the treated RNA is recovered.

13. The method according to any one of claims 6 to 12, wherein more than 70% of the desulphonated RNA is recovered.

14. The method according to claim 13, wherein more than 80% of the desulphonated RNA is recovered.

15. The method according to claim 14, wherein more than 90% of the desulphonated RNA is recovered.

16. The method according to any one of claims 1 to 15, wherein the bisulphite reagent is selected from sodium bisulphite or sodium metabisulphite.

17. The method according to any one of claims 1 to 16, wherein the recovering step is carried out by precipitation of the RNA or by solid phase separation.

18. The method according to claim 17, wherein the recovering step is carried out by precipitation of the RNA.

19. The method according to any one of claims 1 to 18, wherein at least one step is carried out on a solid phase support.

20. The method according to claim 19, wherein the treating, recovering and desulphonation steps are carried out on a solid phase support.

21. The method according to claim 19 or 20, wherein the solid phase support comprises magnetic beads or columns.

22. The method according to any one of claims 6 to 21, wherein the desulphonation step is carried out at a temperature of 40° C.

23. The method according to any one of claims 6 to 22, wherein the desulphonation step is carried out for 5 minutes.

24. The method according to any one of claims 3 to 22, wherein the desulphonation step is carried out at a pH in the range 8.5 to 11.5.

25. The method according to claim 24, wherein the pH is 8.7, 10.5 or 11.5.

26. The method according to claim 7, wherein the alkaline pH is achieved with sodium bicarbonate, Tris-EDTA (TE) buffer or N-cyclohexyl-3-aminopropanesulfonic acid (CAPS) buffer.

27. The method according to any one of claims 1 to 26, wherein the amount of RNA to be treated is 0.5 pg or less.

28. The method according to any one of claims 1 to 26, wherein the amount of RNA to be treated is at least 15-150 attograms.

29. The method according to any one of claims 1 to 28, further comprising a denaturing step prior to the reacting step.

30. A kit for carrying out the method of any one of claims 1 to 29 comprising vessels containing reagents selected from a diluent, a bisulphite reagent and an alkali; and instructions to use the reagents.
